(19) United States
(12) Patent Application Publication
(10) Pub. No.: US 2021/0208149 A1
(43) Pub. Date: Jul. 8, 2021

(54) METHODS FOR CELL PROFILING

(71) Applicant: David Stanley Bailey, Orwell Cambridgeshire (GB)

(72) Inventor: David Stanley Bailey, Orwell Cambridgeshire (GB)

(21) Appl. No.: 17/047,668

(22) PCT Filed: Apr. 15, 2019
(86) PCT No.: PCT/EP2019/059632

§ 371 Date: Oct. 14, 2020

(30) Foreign Application Priority Data
Apr. 16, 2018 (GB) 1806188.7

(51) Int. Cl. G01N 33/574

(52) U.S. Cl. CPC G01N 33/57484

(57) ABSTRACT

This invention relates to methods of characterising mammalian cells, e.g. mammalian cancer cells, based on their chemotypic signature. The methods comprise contacting test mammalian cells with a panel of reference compounds, measuring the effect of each reference compound on a phenotypic property of the test mammalian cells to produce a score for each reference compound in the panel, combining the scores to produce a chemotypic signature, and comparing this signature to the signatures of a set of reference mammalian cell lines. This may be useful for diagnostic and prognostic applications, as well as rational drug design, patient stratification, and personalised drug discovery.

### FIELD

The present invention relates to cell typing, for example in the diagnosis and prognosis of diseases, such as cancer, and the provision of precision therapies.

### BACKGROUND

One of the major challenges in biology is the accurate discrimination of the effects of compounds on tissues and cells, and identifying those compounds showing informative effects to both diagnose and treat underlying disease processes.

One important application of such discrimination is in cancer diagnosis. Many cancers exhibit cellular and histological heterogeneity. For example, one of the most intransigent of cancers, glioblastoma (GBM), displays a high degree of both inter- and intra-tumour heterogeneity [1, 2]. High grade gliomas can be classified into distinct molecular subtypes with different outcomes in terms of disease progression and prognosis [3]. The most common classification of gliomas recognises four distinct subtypes: Proneural, Classical, Mesenchymal, and Neural. In parallel, GBM tumours also exhibit wide diversity at a genetic level [4, 5].

Understanding the molecular subtypes of an individual cancer at a functional level is an essential step in personalising treatments, and for stratifying patients within clinical trials. Specific therapies are likely to be effective against only a defined subset of cells within and between patients' tumours, necessitating personalised cancer treatments [6, 7, 8].

In the development of new targeted therapies, accurate and translational model systems are of utmost importance. For example, a recent initiative from scientists at Uppsala has developed and characterised 48 different GBM cell lines [9], spanning a large part of the heterogeneity observed in GBM patient tumours. Such diverse panels have utility for evaluating targeted treatments.

Traditional cell-based pharmacological screening permits the assessment of the functionality of molecular targets within a natural cellular context. However, specific targets within a cell can show very different activities and functionalities on a cell-by-cell basis, based on the ways in which these targets are biochemically regulated and controlled. Moreover, many molecular targets show differential activities within specific cell types and cell lineages.

Genotyping has been used to type diseases including cancer, to explore personalized medicine. However, the readout of genotyping is not currently directly addressable using pharmacological agents, and the interpretation of genotyping results can be misleading [10] or unpredictable [11].

For cancer, tumour typing approaches that not only allow classification of tumours diagnostically, but also give a direct readout of which therapeutic approaches and which medicines are most likely to be beneficial on a patient to patient basis, would be extremely useful in the provision of precision therapies. This would be particularly useful in cancers such as GBM which show high degrees of heterogeneity.

### SUMMARY

The present inventors have unexpectedly recognised that the specific responses of cells from a patient, such as tumour cells, to a panel of different therapeutic agents can be used to generate a profile or “chemotype” that allows the cells to be diagnostically and prognostically classified and provides predictive information about which drugs and drug combinations are likely to be effective treatments for the patient. The generation of a chemotype may also be useful in rational drug design, patient stratification, and personalised drug discovery, for example in understanding and addressing the processes whereby a patient's tumour cells become drug resistant.

A first aspect of the invention provides a method of characterising mammalian cells comprising;
contacting test mammalian cells with a panel of reference compounds,
measuring the effect of each reference compound in the panel on a phenotypic property of the test mammalian cells to produce a score for each reference compound in the panel,
producing a chemotypic signature or profile for the test mammalian cells from the scores of the individual reference compounds,
comparing the chemotypic signature of the test cells with the phenotypic signatures of a set of reference mammalian cell lines, and
identifying one or more reference cell lines in the set with a signature that corresponds to the signature of the test cells;
wherein the test cells have one or more shared properties with the identified reference cell line.

Phenotypic properties may include the response of the test mammalian cells to specific therapeutic compounds, combinations of therapeutic compounds, or combinations of therapeutic compounds with other therapeutic modalities.

The test mammalian cells may be cancer cells and the reference cell lines may be cancer cell lines.

Other aspects and embodiments of the invention are described in more detail below.

### BRIEF DESCRIPTION OF THE FIGURES

FIG. 1 shows the cross-screening of a single compound, the natural product fucoxanthin, against control (HEK293S) and GBM cell lines (U87 and A172), showing a differential response between the cell types to the compound.

FIG. 2 shows example chemotypes generated using a panel of 50 compounds against a set of 5 GBM cell lines.

FIG. 3 shows some of the potential applications of chemotypic profiling.

### DETAILED DESCRIPTION

This invention is based on the use of the differential sensitivity of a mammalian cell, such as a cancer cell, to a panel of chemical compounds to generate a chemotypic signature or chemotypic profile, briefly referred to as its “chemotype”. The chemotypic signature of closely-related mammalian cells, for example cells within particular developmental lineages, may be determined as described herein and used experimentally to specifically distinguish cells and cell populations. For example, mammalian cells, for example disease cells, such as cancer cells, may be profiled or chemotyped by contacting them with a panel of reference compounds and measuring the precise effect of each reference compound on a phenotypic property, such as viability or proliferation. This measurement generates a score for each reference compound in the panel that is used to produce a chemotypic signature for the test cells.

The chemotypic signature may be compared with the chemotypic signatures of a set of reference cell lines to identify a reference cell line within the set that produces a signature that corresponds to, or is similar to, the signature of the test cells. One or more properties of the test cells, including their differential drug response, can then be predicted from the properties of the identified reference cell line to which they are most similar. This may be useful for example in the stratification of patients and the screening and development of therapeutics.

The test mammalian cells may be human cells. The test mammalian cells may be cells obtained from a patient. For example, the test cells may be obtained from a sample or biopsy of cells or tissue taken from a patient. The patient may have a disease condition, such as cancer.

In some preferred embodiments, the test mammalian cells may be cancer cells and the reference cell lines may be cancer cell lines. The test cancer cells may be cancer cells obtained from a patient. For example, the test cancer cells may be obtained from a sample or biopsy of cancer cells taken from a patient with cancer. The test cancer cells may be tumorigenic cells within the cancer, or non-transformed, associated, supporting cells from the tumour environment around the cancer, or other cells such as immune cells in the environment of the cancer.

Test cells may be prepared from any of the cell types associated with the cancer, and may be prepared using any of the standard, well-characterised techniques available in cell biology, including but not limited to primary cell culture, fluorescent-activated cell sorting, and immune-separation.

The methods described herein may be useful in chemotyping any type of cancer, including skin cancer (in particular melanoma), head and neck cancer, kidney cancer, sarcoma, germ cell cancer (such as teratocarcinoma), liver cancer, lymphoma (such as Hodgkin's or non-Hodgkin's lymphoma), leukaemia (e.g. acute myeloid leukaemia), skin cancer, bladder cancer, breast cancer, uterine cancer, ovarian cancer, prostate cancer, lung cancer, colorectal cancer, cervical cancer, oesophageal cancer, pancreatic cancer, stomach cancer, and cerebral cancer, such as glioblastoma (GBM).

Cancers may be familial or sporadic.

Cancers may be metastatic or non-metastatic.

Suitable cancers may include cancers that allow the provision of cellular and tissue material for example through surgical resection or biopsy.

Cancer is a malignant transformation of mammalian cells. Thus, cerebral cancer, for example, refers to a malignant transformation of cells in the brain. The cancer may be located at its primary location, such as the brain in the case of cerebral cancer, or at a distant location in the case of metastases. A tumour may result from all or any of the cancers mentioned above. For example, a tumour may be the result of a cerebral cancer, such as glioblastoma. A tumour which is the result of a particular cancer may include both a primary tumour and tumour metastases of said cancer. Thus, a tumour which is the result of cancer of a first tissue, for example, includes both a primary tumour in the first tissue and metastases of the cancer found in other tissues of a patient's body.

Mammalian cells are profiled as described herein using a panel of compounds. The mammalian cells are probed with each compound in the panel and the functional response of the cells measured. The different responses of the cells to the different compounds in the panel allow properties of the mammalian cells that relate to certain characteristics, such as viability, migration, radiation sensitivity and proliferative capacity, to be defined and may facilitate the classification of different sub-types or sub-populations of cells.

The number of compounds in the panel may depend on the cellular heterogeneity of the tumour or tissue from which the test cells are derived. A suitable panel may comprise 5 or more, 10 or more, 20 or more, 30 or more, 40 or more, 50 or more, 60 or more, 70 or more, 80 or more, 90 or more, or 100 or more compounds.

In some embodiments, the panel may comprise at least 10 compounds. In other embodiments, for example where chemotyping is being performed on drug resistant cell populations, only a few, alternative chemical agents may be used to obtain an informative chemotype suitable for diagnosis and prognosis.

The compounds in the panel may inhibit one or more of cell proliferation, viability, migration, invasion and angiogenesis or promote apoptosis, radiosensitisation or other detectable property that may be useful in the diagnosis and treatment of a disease, including cancer.

The compounds in the panel are preferably compounds with known (i.e. previously characterised) activity against mammalian cells, such as cancer cells. Suitable compounds include approved drugs and late stage exploratory compounds. For example, a suitable compound for use in a panel may display a reduction of viability, migration, invasion or angiogenesis in at least one reference cell line. Suitable reductions may include at least 10%, at least 20%, at least 30%, at least 40%, or at least 50% or more. Preferably, the compounds in the panel have varying effects on different members of the set of reference cell lines.

Suitable compounds for use in the panel may provide robust discrimination amongst the reference cell lines and may include approved therapeutic compounds, trial and therapeutic compounds and compounds that are related by mode of action to therapeutic compounds. Compounds suitable for use in a panel for a particular disease, tissue or cell type may be selected using preclinical studies focused on that particular disease, tissue or cell type.

In some embodiments, the compounds may alter cell viability, for example the panel may comprise compounds that are cytotoxic to mammalian cells. Suitable compounds may include for example active anti-cancer agents. In other embodiments, the compounds may alter phenotypic properties displayed by mammalian cells, causing cell cycle perturbation, apoptosis enhancement, or changes in antigen display.

A panel of reference compounds may comprise one or more cytotoxic agents and their conjugates, including for example, antitubulin agents, auristatins, DNA intercalators, DNA minor groove binders, DNA replication inhibitors, alkylating agents anthracyclines, antibiotics, antifolates, antimetabolites, chemotherapy sensitizers, duocarmycins, etoposides, fluorinated pyrimidines, ionophores, lexitropsins, nitrosoureas, platinols, platinum complexes such as carboplatin or cisplatin, purine antimetabolites, puromycins, steroids, mitotic inhibitors, topoisomerase inhibitors, angiogenesis inhibitors, Ras antagonists, reactive oxygen species and vinca alkaloids.

A panel of reference compounds may comprise one or more inhibitors, antagonists, activators and agonists of the molecular targets, pathways and processes known to occur in a disease of interest. For example, a GBM panel of reference compounds may comprise inhibitors, antagonists, activators and agonists of one or more of site-1 protease, melanocortin receptor-4, phosphodiesterases, including PDE1, PDE3, PDE4 and PDE10, auxtotaxin, YAP-TEAD complex, PARP, kinases, including GSK-3, focal adhesion kinase, LIM kinases, aurora kinases, cyclin dependent kinases (CDKs) such as CDK1, CDK4, CDK5, CDK6, CDK7 and CDK9, and tyrosine kinases, such as Bruton's tyrosine kinase, SRC tyrosine kinase, and RET-tyrosine kinase; MER, MEK1, MEK2, FLT3, SphK, ErbB, p110-1, IKBKE histone deacetylase, Bcl-2, Bcl-xL, BCL-w, Aurora A kinase, VEGF, PDGF, JMJD3, Eg5, MAP2K, lactate dehydrogenase, glycolysis, transglutaminase 2, STAT3, Hsp90, PI3K, EGFR, mTOR, VEGFR, VEGFR-2, HER2, HDACs, such as HDAC1 and HDAC3, FGFR, PDGFR, c-MET, IGF-1R, PI3K, PKCI, HSP90, mTORC2, microtubules, Mol1, AKT, proteasomes, SERCA, COX, histone methyltransferase, GLI, survivin, carbonic anhydrase, BET, TIE2, TGFBR1, FAK, MAO A, RTL-AXL, G quadruplexes, ROCK, mTORC2, mTORC2, IAP, gamma secretase, NAMPT, MDM2, JAK2, STAT3, ACAT1, PORCN, cap-translation, HSP70, HSP90, telomerase, NF-κβ, HDR, PBK, IGFR1, USP1, BRAF, HER2, integrin, AXL, RNR, ornithine decarboxylase, NEDD8-activating enzyme, ROS1, aromatase, ER and HMG-CoA reductase.

A panel of reference compounds may comprise one or more chemotherapeutic agents, for example alkylating agents such as platinum complexes including cisplatin, mono(platinum), bis(platinum), tri-nuclear platinum complexes and carboplatin, thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU) gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, pitavastatin, fludarabine phosphate, and cladribine; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; binblastine; vindesine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); Topoisomerase inhibitors such as doxorubicin HCl, daunorubicin citrate, mitoxantrone HCl, actinomycin D, etoposide, topotecan HCl, teniposide (VM-26), and irinotecan and pharmaceutically acceptable salts, acids or derivatives of any of the above.

A panel of reference compounds, for example for the characterisation of GBM cells, may comprise one or more compounds shown to be active in preclinical models of GBM. Suitable compounds may include; 2-Amino-3H-phenoxazin-3-one, 2-Hydroxyoleic acid, 3-(3,4-dichlorophenyl)-1-(3,4-dimethylphenyl)-1-(5-methyl-4,5-dihydro-1,3-thiazol-2-yl)urea, 3-Deazaneplanocin, 4egi-1, 5-Nonyloxytryptamine, 6-Hydroxyquinoline-4-carboxylic acid, 7-Ethyl-10-hydroxycamptothecin, 7-Hydroxystaurosporine, 8-Bromo-cyclic AMP, 8-Hydroxy-2-methyl-1H-quinazolin-4-one, 9-ING-41, A-966492, Abemaciclib, Abt-737, AC1MMYR2, Acalabrutinib, Acetazolamide, Adavosertib, AEE788, Afatinib, AG-120, Aldoxorubicin, Alexidine, Alisertib, Altiratinib, Alvespimycin, AMG 232, AMG-925, Amlexanox, Anisomycin, Antroquinonol, Apatinib, Apigenin, Arctigenin, Ardipusilloside 1, Ascochlorin, Astemizole, AT13387, Atorvastatin, Aurintricarboxylic acid, Axitinib, AXL1717, AZD2014, AZD-7451, AZD8055, BAY 11-7082, BDBM86691, Bendamustine, BI2536, Bimiralisib, Binimetinib, Birinapant, BIX-01294, BMS-536924, BMS-777607, Bortezomib, Bosutinib, Bufalin, Buparlisib, Cabazitaxel, Caffeic acid phenethyl ester, Caffeine, Camptothecin, Capecitabine, Capmatinib, Captopril, Carboplatin, Cardamonin, Carmustine, carnosol, Carvacrol, Casticin, Cathepsin S Inhibitor, Caudatin, CBL-0137, CC-115, CC-223, Cediranib, Celastrol, Celecoxib, Ceritinib, Cerivastatin, Chaetocin, Chloroquine, Chlorpromazine, Cilengitide, Cisplatin, Cladribine, Clioquinol, Clofazimine, Clorgiline, Cordycepin, Crenolanib, Crizotinib, Cryptotanshinone, Cucurbitacin I, CUDC-101, Curcumin, Cycloheximide, Cyclophosphamide, D609, Dabrafenib, Dacomitinib, Dactinomycin, Dactolisib, Dasatinib, DB-074971, Demethoxycurcumin, Deoxyglucose, Dexanabinol, Dibutyryl cAMP, Diclofenac, Digitoxigenin, Digitoxin, Digoxigenin, Digoxin, Dihydroartemisinin, Dimethylaminomicheliolide, Dinaciclib, Disufenton sodium, Disufenton sodium, Docetaxel, Dorsomorphin, Dovitinib, Doxazosin, Doxorubicin, Eflornithine, Embelin, Enasidenib, Entrectinib, Enzastaurin, Epigallocathecin, Epigallocathecin Gallate, Epirubicin, Erdafitinib, Erlotinib, Etoposide, Everolimus, Evodiamine, Evofosfamide, Farnesylthiosalicylic acid, Fasudil, Fenofibrate, Fingolimod, Fingolimod, Flavopiridol, Flubendazole, Fluorouracil, Fluvoxamine, Foretinib, Forskolin, Fotemustine, Fucoxanthin, Galunisertib, Gamma-Secretase Inhibitor IX, GANT61, Gartanin, GDC-0084, GDC-0941, Gedatolisib, Gefitinib, Genistein, Germacrone, GK921, Glasdegib, Glaucocalyxin A, Golvatinib, Gossypol, GSK J4, GSK1838705A, GSK1904529A, GSK3326595, GSK461364, Guggulsterone, Hispolon, Honokiol, Hsp-990, Hydroxyurea, Hypericin, Ibrutinib, Ibuprofen, Idarubicin, Imatinib, Imiquimod, Indatraline, Indirubin Derivative E804, Indomethacin, Infigratinib, Iniparib, Irinotecan, Isobutylmethylxanthine, Isoliquiritigenin, Isotretinoin, Itraconazole, Ixazomib, JQ1, JS-K, Juglone, Karenitecin, KPT251, KPT276, KU55933, KU-55933, KU60019, Lactacystin, Lanatoside C, Lapatinib, Larotrectinib, LB-100, Lenalidomide, Lenalidomide, Lenvatinib, LEQ506, Letrozole, LGK974, Lomustine, Lonafarnib, Lorlatinib, Lovastatin, LOXO-195, Luteolin, LY294002, LY341495, Marizomib, MAZ51, Mebendazole, Melatonin, Mepacrine, Metformin, Methotrexate, Methyl gallate, MG-132, Mibefradil, Midazolam, Mitoxantrone, MK-2206, ML00253764, MLN4924, Monensin, MRK003, Naringin, Navitoclax, NBQX, Nelfinavir, Neratinib, Niclosamide, Nilotinib, Nintedanib, Nisoldipine, NKTR-102, NMS-P715, Nobiletin, NSC141562, NSC23766, NVP-AEW541, NVX-207, Olanzapine, Olaparib, Oleuropein, Oligomycin, Omacetaxine, ON123300, Oroxindin, Oroxylin A, Otx-008, OTX-015, Ouabain, Oxaliplatin, Oxamate, Paclitaxel, Palbociclib, Palomid 529, Pamiparib, Panobinostat, Pazopanib, PCI-24781, PD0325901, PD173074, Pegdinetanib, Perifosine, Perilla alcohol, Pexidartinib, PF-04691502, PF-06840003, PF-429242, PF8380, Phenformin, PI-103, Pimozide, Piplartine, Pitavastatin, PJ34, Plumbagin, PLX-4720, Pomolic acid, Ponatinib, PP242, PQ401, Pregnenolone, Procarbazine, Propofol, Proscillaridin A, Punicalagin, PX-866, Pyrvinium, Quercetin, R428, Ralimetinib, RapaLink-1, Regorafenib, Repaglinide, Resveratrol, Reversan, RG2833, RG7112, Ribociclib, Riluzole, Ritonavir, R04929097, Rolipram, Romidepsin, Roscovitine, Rottlerin, Rucaparib, Ruxolitinib, Salinomycin, Salvianolic acid B, Sanguinarine, Satraplatin, Savolitinib, Schizandrin B, Scriptaid, Selinexor, Selumetinib, Semustine, Sepantronium bromide, Sertraline, Sevoflurane, SF1126, SGX-523, SH-4-54, SH5-07, Shikonin, Silibinin, Silmitasertib, Sinomenine, Sirolimus, SKI II, Sonidegib, Sorafenib, SSR128129E, STX-0119, Sulfasalazine, Sunitinib, T56-LIMKi, Talampanel, Tamoxifen, Tandutinib, Tanespimycin, Telomestatin, Temozolomide, Temsirolimus, Terfenadine, Tesevatinib, Tetraethylammonium, Tetrandrine, TG02, TG101209, TGX-221, Thalidomide, Thapsigargin, Theobromine, Thymoquinone, THZ1, Tipifarnib, Tivozanib, Tnp-470, Tocladesine, Topotecan, Tozasertib, Tpi-287, Trametinib, Transcrocetinate sodium, Tricetin, Trichostatin, Trifluoperazine, Triptolide, UMI-77, UNC2025, UNII-2AW48LAZ41, USL311, VAL-083, Valproic Acid, Vandetanib, Vatalanib, Veliparib, Vemurafenib, Vinblastine, Vincristine, Vindesine, Vinorelbine, Vinpocetine, Vismodegib, Visudyne, Vorinostat, Wortmannin, WP1066, WP1130, XL-184, XL765, Xyloketal B, YU238259, ZSTK474.

The composition of the panel may vary depending on the nature of the test mammalian cells. For example, the panel may vary depending on the type of cancer from which the test cells are derived. In some embodiments, the size of the panel may depend on the heterogeneity of the test mammalian cells. For example, the panel may be larger for more heterogeneous cancers, e.g. GBM and colorectal cancer. Panel size may also vary for example between primary and metastatic cancers.

Panels of compounds may be used to chemotype cells associated with non-cancer diseases. For example, a panel of compounds might be useful in indicating new patterns of drug response in hormone-dependent conditions. For example, a panel of compounds may be useful in indicating new patterns of drug response in diabetes using insulin secretion as a phenotypic readout.

The compounds in the panel may alter a phenotypic property of mammalian cells. For example, the compounds may reduce or inhibit the proliferation, viability, migration, invasion and/or angiogenesis of mammalian cells, such as cancer cells; increase or promote apoptosis and/or radiosensitisation; and/or may alter a cell surface phenotype, such as EGFRvIII or other neoantigen, or a molecular characteristic associated with differentiation and apoptotic processes, antigen display and cell renewal.

Suitable phenotypic properties for use in generating a chemotypic signature may include any parameter for which assays are available, including diagnostic assays for a particular disease, such as antibody production in multiple myeloma, or insulin hormone production in diabetes. The phenotypic property may be measured using any convenient technique. For example, proliferation and/or viability may be measured using a viability assay. Suitable viability assays are available from commercial suppliers (e.g. Alamar Blue, Serotec; CCK8, Merck).

The effect of a compound on the phenotypic property of the test mammalian cells, such as cancer cells, is measured and used to produce a score. The score represents the measured extent of the change in the phenotypic property of the test cells in response to the compound.

For example, the score may be the amount of inhibition of proliferation or reduction in viability of the test cells by the compound. Conveniently, the score for the effect of a compound from the panel on the test cells may be expressed as an EC50 or IC50, or expressed as % inhibition of a specific phenotypic endpoint.

The chemotypic signature or profile of a mammalian cell is an array of the scores for the responses of the mammalian cell to the different compounds in the panel. The chemotypic signature may be expressed graphically, numerically or in any other form.

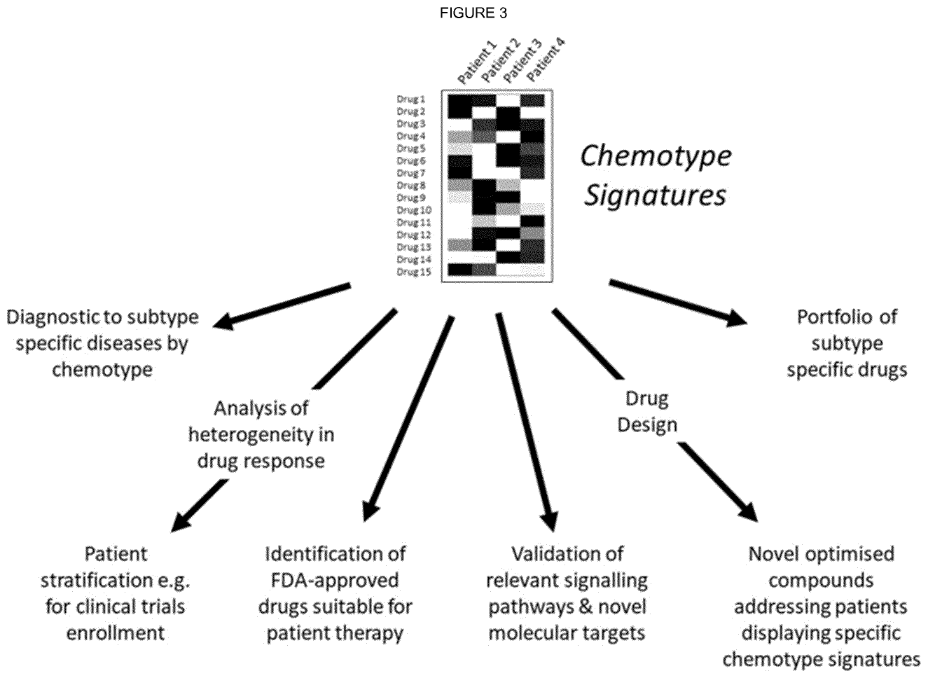

The chemotypic signature or profile may be indicative of the biological processes that perform specific physiological functions in cells and cell lineages, based on their pharmacology, biochemistry and degree of cellular differentiation. At a diagnostic level, the chemotypic signature or profile of a mammalian cell may also be indicative of the cell's ability to be altered or modified by treatment with one or more therapeutic compounds. A chemotypic signature or profile may be indicative of resistance or sensitivity to such treatment. For example, a chemotypic signature or profile may be predictive of patient response to a compound in the panel, a combination of compounds in the panel or a compound not present in the panel. This may be useful in stratifying patients suitable for treatment with a compound or in the development of compounds suitable for treating patient populations, as outlined in FIG. 3.

A test mammalian cell may be characterised by comparing its chemotypic signature with the chemotypic signatures of a set of reference cell lines generated using the same panel of compounds.

The set may comprise 5 or more, 10 or more, 20 or more, 30 or more, 40 or more, 50 or more or 100 or more different reference cell lines.

The reference cell lines may be of the same cell type or lineage as the test mammalian cells. In some embodiments, the reference cancer cell lines may be of the same cancer type as the test cancer cells. For example, the reference and test cancer cells may both be glioblastoma cells.

Reference cell lines in the set may be heterogeneous and may include cell lines of different lineages or sub-types, different properties, different diagnoses or prognoses and/or different sensitivities to anti-cancer compounds. For example, reference cancer cell lines in the set may include cell lines of different sub-types of the cancer, cell lines of the cancer with different properties, such as invasiveness, cell lines of the cancer with different diagnoses or prognoses and cell lines of the cancer with different sensitivities to anti-cancer compounds. The reference cancer cell lines in the set may display different sensitivities to the compound in the panel. Preferably, the set of reference cancer cell lines is representative of the range of heterogeneity of the cancer in patient populations.

The properties, features, characteristics, prognoses, and sensitivities of the reference cancer cell lines in the set may be predetermined.

Reference cell lines for use in characterising test cells from a disease, tissue or cell type may be derived from that disease, tissue or cell type. For example, a set of reference cell lines for characterising GBM may be derived from selected brain tumours from diagnosed glioblastoma patients, provided that they can be stably maintained in culture.

In some embodiments, the reference cell lines may include established cell lines, primary patient-derived cell lines or component cells identifiable within disease-associated organoids. For example, the cell lines may be immortalised human cancer cell lines, stem-cell like primary cancer cells or primary patient-derived cancer lines grown as adherent cultures. Suitable reference cell lines may be obtained from commercial culture collections (e.g. ATCC) or generated from primary cells obtained from a patient.

Suitable reference cell lines for use in a panel of cancer cell lines or glioblastoma cell lines may include primary human cancer cells, such as low-passage patient-derived glioma and glioblastoma cell lines, or established cell lines such as the well characterised cell lines A172, LN18, LN229, LNZ308, T98G, U118, U138, U251, U343, U373 and U87, or selected glioma and glioblastoma cell lines from more extensive cell culture collections such as the well-known ATCC and ECACC cell collections, as well as cell collections dedicated to GBM such as the HGCC.

The signatures of the set of reference cell lines may be produced by a method comprising;
contacting a reference cell line from the set with the panel of compounds,
measuring the effect of each compound of said panel on a phenotypic property of the reference cell line in the set to produce a score for each compound in the panel,
producing a chemotypic signature for the reference cell line from the scores of the panel; and
repeating the above steps to produce signatures for all the reference tumour cell lines in the set.

The signatures of the set of reference cells may be in the form of a matrix or table and may be expressed graphically, numerically, digitally or in any other way that allows comparative assessment.

The overall, complete heterogeneity of a particular disease may be defined as the point at which the chemotypic signatures obtained from the set of reference cells using the panel of compounds defines all the drug responses observed within the tested clinical isolates of the disease.

The signature of the test mammalian cells may be compared to the signatures of the set of reference cell lines. Reference cell lines with signatures that match the test cell signature showing the highest similarities in chemotype signatures are identified. Signature matching and stratification may be performed using informatics and machine learning approaches that are established in the art.

Test cells with a signature that corresponds to or matches the signature of a reference cell line may have one or more common properties or characteristics with the reference cell line. For example, the test cell may be of the same sub-type as the reference cell line; display similar properties, such as invasiveness; have a similar prognosis or a similar sensitivity to one or more therapeutic compounds, such as anti-cancer compounds.

The method may further comprise predicting from the identified reference cell line the responsiveness of the test cells to a therapeutic compound or a combination of therapeutic compounds. For example, a therapeutic compound or combination to which the test cells are sensitive may be identified.

A chemotype signature as described herein provides an accurate, informative subtyping of a sample of mammalian cells, for example cancer cells from a patient's tumour. From data on treatment outcomes from patients, a database of tumour subtype specific treatment options may be compiled and used to guide treatment and provide accurate prognoses following subtyping using the chemotype signature.

A chemotype signature as described herein provides a detailed context in which the comparative performance of therapeutic compounds can be assessed. To establish relevant chemotypic signatures, therapeutic compounds can be specifically administered singly or in combination, and outcomes evaluated at an early stage of iterative treatments, for example within adaptive clinical trials.

Cumulative experience with chemotype signatures may predict future clinical outcome, and may allow the identification of a treatment, for example, an anti-cancer compound, combination of compounds, or regimen that is specific for the patient from whom the test cells were obtained i.e. to which the patient's cancer or other condition is responsive.

A chemotype signature obtained from a sample of test cells may be predictive of either a particular response, or patient clinical outcomes, leading to a stratification of patient drug responses or definition of their prevailing molecular and cellular subtypes, reflecting a differential cell line response.

In some embodiments, a method may comprise identifying a population of patients who are predicted to be responsive to a therapeutic compound or a combination of therapeutic compounds. This may be useful, for example, in stratifying patients for clinical trials.

In other embodiments, a method may comprise identifying a therapeutic compound or a combination of therapeutic compounds that is predicted to be effective in a population of patients. This may be useful, for example in developing drug candidates that are effective within large patient populations.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Tables

Table 1 shows some selected compounds showing differential effects in a panel of reference cell lines.

Table 2 summarises screening results generated using a panel of 15 compounds against a set of reference cell lines, in this case GBM cell lines. The EC50 values (μM) are shown. The cumulative set of EC50 values provides a unique signature or chemotype for each cell line.

### EXPERIMENTAL

A wide range of approved and exploratory drugs are profiled for their ability to inhibit GBM cell proliferation. Their EC50 values against each separate cell line in the set are determined. Initial cell-based proliferation assays are carried out using a cell counting kit (CCK8, product number 96992, Merck) using established protocols. Screening is performed in 96-well or 384-well or 1536-well formats.

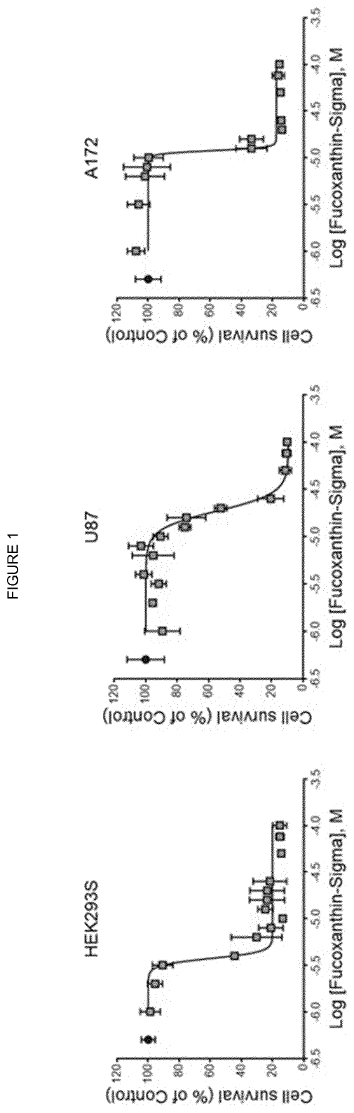

An example of a set of EC50 determinations for fucoxanthin (Sigma-Aldrich Corporation), a naturally-occurring xanthophyll with potential pan-cancer therapeutic activity [12] is shown in FIG. 1.

Some additional compounds suitable for inclusion in a chemotyping panel are shown in Table 1. These include examples of pharmacological probes, such as trequinsin, natural products such as fucoxanthin, exploratory drugs such as PF04449613, drugs in clinical trials, such as PF02545920, TIC10, PF04691502, GSK1059615 and LY294002, and FDA approved drugs, such as Axitinib and Temozolomide. Data was obtained from testing these compounds at fixed concentrations against normal control cells (HEK293S) alongside reference GBM cells (U87 and A172), to obtain a dataset (Table 1) within which to compare the pharmacological and pharmaceutical properties of compounds across a chosen chemotyping panel.

The conversion of differential drug responses into a chemotyping matrix using established GBM cell lines is exemplified in Table 2, adapted from reference [13]. The EC50 values (μM) of a panel of 15 drugs against different GBM cell lines are shown. The cumulative set of EC50 values provides a unique signature or chemotype for each cell line.

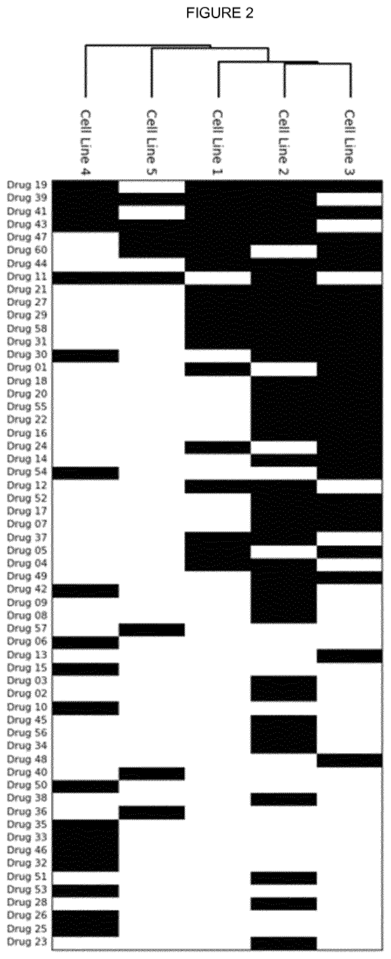

A detailed chemotype derived from the responses of 5 cell lines (x axis) to a panel of 50 compounds (y axis) is shown in FIG. 2. The responses of the cell lines to the panel can be clustered from their chemotype profiles, and drugs that are likely to be effective or ineffective against the different clusters predicted.

TABLE 1
Compounds tested for potential inclusion in the chemotyping panel

| | | Test Concentration |
|---|---|---|
| Compound | Description | log[M] |
| Trequinsin | Pharmacological Probe | −4.6 |
| Fucoxanthin (Sigma) | Natural Product | −4.8 |
| PF04449613 | Exploratory Compound | −4 |
| PF02545920 | In Clinical Trials | −4.6 |
| TIC10 | In Clinical Trials | −5 |
| PF04691502 | In Clinical Trials | −6.6 |
| GSK1059615 | In Clinical Trials | −5.5 |
| LY294002 | In Clinical Trials | −5 |
| Axitinib | FDA Approved Drug | −5 |
| Temozolomide | FDA Approved Drug | −5 |

### Data Obtained from Screening Compounds within a Chemotyping Panel Against a Control Cell Line (HEK293S) and Two Reference GBM Cell Lines (U87 and A172)

| | % Proliferation | | | % Inhibiton | | |
|---|---|---|---|---|---|---|
| Compound | HEK293 | U87 | A172 | HEK293 | U87 | A172 |
| Trequinsin | 41 | 89 | 63 | 59 | 11 | 37 |
| Fucoxanthin | 24 | 74 | 34 | 76 | 26 | 66 |
| PF04449613 | 90 | 114 | 39 | 10 | −14 | 61 |
| PF02545920 | 16 | 70 | 20 | 84 | 30 | 80 |
| TIC10 | 17 | 73 | 32 | 83 | 27 | 68 |
| PF04691502 | 55 | 65 | 56 | 45 | 35 | 44 |
| GSK1059615 | 21 | 43 | 26 | 79 | 57 | 74 |
| LY294002 | 98 | 117 | 58 | 2 | −17 | 42 |
| Axitinib | 103 | 86 | 20 | −3 | 14 | 80 |
| Temozolomide | 100 | 95 | 87 | 0 | 5 | 13 |

TABLE 2

| | | | | | | | |
|---|---|---|---|---|---|---|---|
| U87 | A172 | LN443 | U118 | U87 | A172 | LN443 | U118 |

| | | | | |
|---|---|---|---|---|
| Triptolide | 0.006532 | 0.008329 | 0.03076 | 0.01008 |
| Homoharringtonin | 0.022 | N.A. | 0.02889 | 1.1419 |
| Dactinmycin | 0.4368 | 0.09932 | 0.01958 | 0.05681 |
| Docorubicine | 0.05512 | 0.04877 | 0.06286 | 0.04107 |
| Epirubicin | 0.05927 | 0.06217 | 0.04123 | 0.04687 |
| Topetecan | 0.167 | 0.6493 | 0.1358 | 0.184 |
| Itraconazole | 0.389 | 6.974 | >20 | 1.458 |
| Vindesine sulfate | 0.414 | 0.02317 | 0.4948 | 0.67 |
| Cericastatin | 0.6683 | 0.03166 | 0.02404 | 0.741 |
| Vincrinstine | 1.317 | 0.04202 | 0.8726 | 1.194 |
| Deoxyadenosine | 2.113 | 1.769 | N.A. | 0.5897 |
| Pitavastatin | 3.517 | 0.2737 | 0.083 | 1.923 |
| Sertraline | 4.871 | 3.376 | 6.05 | 3.857 |
| Irinitecan | 8.027 | 7.967 | 4.013 | 4.596 |
| Clofaximine | 8.609 | 11.91 | 20.14 | 19.67 |

### REFERENCES

1. Ricard D, Idbaih A, Ducray F, et al (2012) Primary brain tumours in adults. Lancet 379:1984-1996. doi: http://doi.org/10.1016/S0140-6736(11)61346-9
2. Sottoriva A, Spiteri I, Piccirillo S G M, et al (2013) Intratumor heterogeneity in human glioblastoma reflects cancer evolutionary dynamics. Proc Natl Acad Sci USA 110:4009 LP-4014
3. Phillips H S, Kharbanda S, Chen R, et al (2006) Molecular subclasses of high-grade glioma predict prognosis, delineate a pattern of disease progression, and resemble stages in neurogenesis. Cancer Cell 9:157-173. doi: https://doi.org/10.1016/j.ccr.2006.02.019
4. Brennan C W, Verhaak R G W, McKenna A, et al (2013) The Somatic Genomic Landscape of Glioblastoma. Cell 155:462-477. doi: https://doi.org/10.1016/j.cell.2013.09.034
5. Verhaak R G W, Hoadley K A, Purdom E, et al (2010) Integrated Genomic Analysis Identifies Clinically Relevant Subtypes of Glioblastoma Characterized by Abnormalities in PDGFRA, IDH1, EGFR, and NF1. Cancer Cell 17:98-110. doi: https://doi.org/10.1016/j.ccr.2009.12.020
6. Schilsky R L (2010) Personalized medicine in oncology: the future is now. Nat Rev Drug Discovery 9:363
7. Oh Y T, Cho H J, Kim J, et al (2014) Translational Validation of Personalized Treatment Strategy Based on Genetic Characteristics of Glioblastoma. PLoS One 9:e103327
8. Olar A, Aldape K D (2014) Using the molecular classification of glioblastoma to inform personalized treatment. J Pathol 232:165-177. doi: 10.1002/path.4282
9. Xie Y, Bergstrôm T, Jiang Y, et al (2015) The Human Glioblastoma Cell Culture Resource: Validated Cell Models Representing All Molecular Subtypes. EBioMedicine 2:1351-1363. doi: https://doi.org/10.1016/j.ebiom.2015.08.026
10. Voest E E, Bernards R (2016) DNA-Guided Precision Medicine for Cancer: A Case of Irrational Exuberance? Cancer Discov. 6:130-2.
11. Niepel M, Häfner M, Duan Q, et al (2017) Common and cell-type specific responses to anti-cancer drugs revealed by high throughput transcript profiling. Nat Commun 8:1186. doi: 10.1038/s41467-017-01383-w
12. Satomi Y (2017) Antitumor and Cancer-preventative Function of Fucoxanthin: A Marine Carotenoid. Anticancer Res. 37:1557-1562.
13. Jiang P, Mukthavavam R, Chao Y, et al (2014) Novel anti-glioblastoma agents and therapeutic combinations identified from a collection of FDA approved drugs. J Trans Med 12:13. doi: 10.1186/1479-5876-12-13

1. A method of characterising mammalian cells comprising;
contacting test mammalian cells with a panel of reference compounds,
measuring the effect of each reference compound in the panel on a phenotypic property of the test mammalian cells to produce a score for each reference compound in the panel,
producing a chemotypic signature for the test mammalian cells from the scores of the reference compounds,
comparing the phenotypic signature of the test mammalian cells with the chemotypic signatures of a set of reference mammalian cell lines, and
identifying a reference cell line in the set with a signature that corresponds to the signature of the test mammalian cells;
wherein the test mammalian cells have one or more shared properties with the identified reference mammalian cell line.

2. A method according to claim 1 wherein the test mammalian cells are human cells.

3. A method according to claim 1 wherein the test mammalian cells are obtained from a patient sample.

4. A method according to claim 1 wherein the test mammalian cells and the reference mammalian cell lines are from the same disease or tissue type.

5. A method according to claim 1 wherein the panel comprises 5 or more compounds.

6. A method according to claim 1 wherein the compounds in the panel inhibit one or more of cell proliferation, viability, migration, invasion and angiogenesis or promote apoptosis or radiosensitisation.

7. A method according to claim 1 wherein the compounds in the panel are cytotoxic compounds.

8. A method according to claim 1 wherein the compounds in the panel are anti-cancer agents.

9. A method according to claim 1 wherein the phenotypic property is viability, migration, invasion and angiogenesis or promote apoptosis or radiosensitisation.

10. A method according to claim 9 wherein the phenotypic property is proliferation.

11. A method according to claim 10 wherein proliferation is determined by measuring the number of viable test mammalian cells following contact with the compound.

12. A method according to claim 1 wherein the score is an EC50 value.

13. A method according to claim 1 wherein the set of reference cell lines comprises 5 or more cell lines.

14. A method according to claim 1 wherein the reference cell lines in the set display chemotypic signatures.

15. A method according to claim 1 wherein the set of reference cell lines is representative of the range of heterogeneity of the mammalian cell in patient populations.

16. A method according to claim 1 wherein the signatures of the set of reference mammalian cell lines are produced by a method comprising;
contacting a reference cell in the set with the panel of compounds,
measuring the effect of each compound of said panel on a phenotypic property of the reference cell line in the set to produce a score for each compound in the panel,
producing a signature for the reference cell line from the scores of the panel; and
repeating the above steps to produce a signature for each of the reference cell lines in the set.

17. A method according to claim 1 comprising diagnosing the test mammalian cells as the same sub-type, class or category as the identified reference cell line.

18. A method according to claim 1 further comprising predicting from the identified reference cell line the responsiveness of the test mammalian cells to a compound or a combination of compounds.

19. A method according to claim 1 wherein the test mammalian cells are cancer cells and the reference mammalian cell lines are reference cancer cell lines.

20. A method according to claim 1 wherein the test cancer cells and the reference cancer cell lines are from the same cancer type.

21. A method according to claim 20 wherein the test cancer cells and the reference cancer cell lines are from glioblastoma.

22. A method according to claim 19 wherein the set of reference cancer cell lines is representative of the range of heterogeneity of the cancer type in patient populations.

23. A method according to claim 19 wherein the signatures of the set of reference cancer cell lines are produced by a method comprising;
contacting a reference cancer cell line in the set with the panel of compounds,
measuring the effect of each compound of said panel on a phenotypic property of the reference cancer cell line in the set to produce a score for each compound in the panel,
producing a signature for the reference cancer cell line from the scores of the panel; and
repeating the above steps to produce signatures for all the reference cancer cell lines in the set.
